(19) United States
(12) Patent Application Publication
(10) Pub. No.: US 2025/0304687 A1
(43) Pub. Date: Oct. 2, 2025

(54) MULTISPECIFIC ANTIBODIES TARGETING CD79B/CD3 AND THE USES THEREOF

(71) Applicant: LTZ Therapeutics Inc., Redwood City, CA (US)

(72) Inventors: Jinze Li, Danville, CA (US); Jianhui Zhou, Fremont, CA (US); Su Huang, Rochester, MN (US); Zhentao Sheng, Sunnyvale, CA (US)

(21) Appl. No.: 18/625,012

(22) Filed: Apr. 2, 2024

(51) Int. Cl. C07K 16/28; A61P 35/00; G01N 33/68

(52) U.S. Cl. CPC C07K 16/2809; A61P 35/00; G01N 33/6854; C07K 2317/31; C07K 2317/526; C07K 2317/55; C07K 2317/565; C07K 2317/622; G01N 2333/7051; G01N 2333/70596

(57) ABSTRACT

The disclosure relates to a multispecific (e.g., bispecific) antibody, which includes a first antigen-binding domain that specifically binds to a first antigen and a second antigen-binding domain that specifically binds to a second antigen. The first antigen is CD79b and the second antigen is not CD79b. The disclosure also relates to nucleic acid molecules, vectors and host cells encoding the bispecific antibodies, derivatives of the bispecific antibodies, and their use for disease treatment.

### REFERENCE TO SEQUENCE LISTING SUBMITTED ELECTRONICALLY

This application contains a Sequence Listing that has been submitted electronically as an XML file named “55679-0006001_SL_ST26.XML.” The XML file, created on Apr. 2, 2024, is 67,233 bytes in size. The material in the XML file is hereby incorporated by reference in its entirety.

### TECHNICAL FIELD

The disclosure relates to the field of biomedicine. Specifically, the disclosure relates to a multispecific (e.g., bispecific) antibody, which comprises a first antigen-binding domain that specifically binds to a first antigen and a second antigen-binding domain that specifically binds to a second antigen. The first antigen is CD79b, and the second antigen is not CD79b. The disclosure also relates to nucleic acid molecules, vectors and host cells encoding the bispecific antibodies, derivatives of the bispecific antibodies, and their use for disease treatment.

### BACKGROUND

CD79 is a heterodimeric molecule consisting of CD79a and CD79b. CD79 is expressed almost exclusively on B cells and B cell tumors. As a signaling component of the B cell antigen receptor (BCR), CD79 forms a BCR complex together with the cell surface immunoglobulin (sIg) used for antigen recognition and plays a key role in B cell maturation and activation. Both CD79a and CD79b contain a single extracellular Ig domain, a transmembrane domain, and an intracellular signaling domain, which initiate BCR signaling upon antigen binding, ultimately leading to B cell activation, antigen presentation, cytokine production, cell proliferation, and differentiation. During B cell ontogeny, expression of CD79a and CD79b precedes immunoglobulin (Ig) heavy chain gene rearrangement and expression of CD20 and disappears later than CD20 during late (plasma cell) stages of B cell differentiation. Therefore, antibodies targeting CD79a and CD79b can be used to differentiate B-cell tumors from T-cell tumors or myeloid tumors, or to differentiate L&H lymphocyte-predominant Hodgkin lymphoma from classical Hodgkin lymphoma. Furthermore, anti-CD79a and anti-CD79b antibodies are effective markers for the diagnosis of precursor B-acute lymphoblastic leukemia (pre-B-ALL) because these tumors are negative for other B-cell markers such as CD20 and CD45RA.

Antigen binding to CD79 rapidly induces endocytosis and delivery to the major histocompatibility complex class II (MHCII) compartment (i.e., the lysosomal compartment for class II antigen presentation by B cells). This unique intracellular transport makes CD79 a potential target for cell-targeted delivery of toxic agents, because toxic agents can be delivered directly to target cells into the lysosomal compartment, enhancing cytotoxic activity. This approach also allows the use of more stable linkers that are cleaved in the MHCII compartment. In addition, CD79 is considered a therapeutic target for antibodies because it is physiologically specific in mature B cells and the majority of B-cell non-Hodgkin's lymphomas (B-NHLs). Other cancers expressing CD79 include diffuse large B-cell lymphoma (DLBCL) (90-100%), acute B-lymphoblastic leukemia (B-ALL), chronic lymphocytic leukemia (CLL), B-cell prelymphocytic leukemia (PLL), splenic lymphoma of villous lymphocytes (SLVL), hairy cell leukemia (HCL), follicular lymphoma (FL), and mantle cell lymphoma (MCL).

The CD3 receptor complex is a protein complex composed of four chains. In mammals, the complex contains one CD3y (γ) chain, one CD35 (δ) chain, and two CD3e (ε) chains. These chains bind to the T cell receptor (TCR) and the so-called ζ (ζ) chain to form the T cell receptor CD3 complex and generate an activation signal in T lymphocytes. The CD3y (γ), CD35 (δ), and CD3e (ε) chains are highly related cell surface proteins of the immunoglobulin superfamily that contain a single extracellular immunoglobulin domain. The intracellular tail of the CD3 molecule contains a single conserved motif called the immunoreceptor tyrosine activation motif, or ITAM, which is critical to the signaling ability of TCR.

In recent years, bispecific antibodies (bsAbs) that specifically recognize tumor-associated antigens and the T-cell antigen CD3 have shown efficacy in treating cancer. By bringing tumor cells and T cells together, they can trigger activation and proliferation of T cells, which release cytotoxic molecules such as granzymes and perforin, inducing tumor cell lysis. Bispecific antibodies specific for T cell receptors have higher therapeutic potential than monospecific antibodies.

### SUMMARY

After in-depth research, the inventors constructed multispecific (e.g., bispecific) antibodies that can target CD79b and CD3 at the same time. These CD79b/CD3 multispecific (e.g., bispecific) antibodies can be used to treat CD79b related diseases (e.g., B-cell lymphoma). These CD79b/CD3 multispecific (e.g., bispecific) antibodies can connect cytotoxic T cells with cancer cells, leading to cytotoxic T cell mediated cancer cell killing.

### Bispecific Antibody

In one aspect, the invention provides a multispecific (e.g., bispecific) antibody comprising a first antigen-binding domain that specifically binds a first antigen and a second antigen-binding domain that specifically binds a second antigen, the first antigen is CD79b, and the second antigen is not CD79b.

In some embodiments, the first antigen-binding domain comprises a first heavy chain variable region (VH) and a first light chain variable region (VL) and the second antigen-binding domain comprises a second VH and a second VL.

In some embodiments, the first VH comprises CDR1 comprising the amino acid sequence set forth in SEQ ID NO: 3, CDR2 comprising the amino acid sequence set forth in SEQ ID NO: 4, and CDR3 comprising the amino acid sequence set forth in SEQ ID NO: 5; and the first VL comprises CDR1 comprising the amino acid sequence set forth in SEQ ID NO: 6, CDR2 comprising the amino acid sequence set forth in SEQ ID NO: 7, and CDR3 comprising the amino acid sequence set forth in SEQ ID NO: 8;
preferably, the CDRs are defined by the Kabat numbering system.

In certain embodiments, the first antigen-binding domain comprises a first VH comprising the amino acid sequence set forth in SEQ ID NO: 9 or its variant thereof, and a first VL comprising the amino acid sequence set forth in SEQ ID NO: 10 or its variant thereof;
wherein the variant comprises an amino acid sequence that is at least 80%, 85%, 90%, 91%, 92%, 93%, 94%, 95%, 96%, 97%, 98%, 99%, or 100% identical to original sequence, or has one or several amino acid substitutions, deletions, or additions (e.g., 1, 2, 3, 4 or 5 amino acid substitutions, deletions or additions) compared with original sequence; preferably, the substitutions are conservative substitutions.

In certain embodiments, the first antigen-binding domain comprises a first VH comprising the amino acid sequence set forth in SEQ ID NO: 9, and a first VL comprising the amino acid sequence set forth in SEQ ID NO: 10.

In certain embodiments, the second antigen is selected from CD3, CD19, CD20, CD32B, CD137, CTLA-4, or BCMA.

In certain embodiments, the second antigen is CD3.

In certain embodiments, the second antigen-binding domain comprises a second heavy chain variable region (VH) and a second light chain variable region (VL), wherein,
the second VH comprises CDR1 comprising the amino acid sequence set forth in SEQ ID NO: 13, CDR2 comprising the amino acid sequence set forth in SEQ ID NO: 14, and CDR3 comprising the amino acid sequence set forth in SEQ ID NO: 15; and the second VL comprises CDR1 comprising the amino acid sequence set forth in SEQ ID NO: 16, CDR2 comprising the amino acid sequence set forth in SEQ ID NO: 17, and CDR3 comprising the amino acid sequence set forth in SEQ ID NO: 18;
the second VH comprises CDR1 comprising the amino acid sequence set forth in SEQ ID NO: 22, CDR2 comprising the amino acid sequence set forth in SEQ ID NO: 23, and CDR3 comprising the amino acid sequence set forth in SEQ ID NO: 24; and the second VL comprises CDR1 comprising the amino acid sequence set forth in SEQ ID NO: 25, CDR2 comprising the amino acid sequence set forth in SEQ ID NO: 26, and CDR3 comprising the amino acid sequence set forth in SEQ ID NO: 27; or
the second VH comprises CDR1 comprising the amino acid sequence set forth in SEQ ID NO: 51, CDR2 comprising the amino acid sequence set forth in SEQ ID NO: 52, and CDR3 comprising the amino acid sequence set forth in SEQ ID NO: 53; and the second VL comprises CDR1 comprising the amino acid sequence set forth in SEQ ID NO: 54, CDR2 comprising the amino acid sequence set forth in SEQ ID NO: 55, and CDR3 comprising the amino acid sequence set forth in SEQ ID NO: 56;
preferably, the CDRs are defined by the Kabat numbering system.

In certain embodiments, the second antigen-binding domain comprises a second VH comprising the amino acid sequence set forth in SEQ ID NO: 19 or its variant thereof, and a second VL comprising the amino acid sequence set forth in SEQ ID NO: 20 or its variant thereof;
the second antigen-binding domain comprises a second VH comprising the amino acid sequence set forth in SEQ ID NO: 28 or its variant thereof, and a second VL comprising the amino acid sequence set forth in SEQ ID NO: 29 or its variant thereof; or
the second antigen-binding domain comprises a second VH comprising the amino acid sequence set forth in SEQ ID NO: 57 or its variant thereof, and a second VL comprising the amino acid sequence set forth in SEQ ID NO: 58 or its variant thereof;
wherein the variant comprises an amino acid sequence that is at least 80%, 85%, 90%, 91%, 92%, 93%, 94%, 95%, 96%, 97%, 98%, 99%, or 100% identical to original sequence, or has one or several amino acid substitutions, deletions, or additions (e.g., 1, 2, 3, 4 or 5 amino acid substitutions, deletions or additions) compared with original sequence; preferably, the substitutions are conservative substitutions.

In certain embodiments, the second antigen-binding domain comprises a second VH comprising the amino acid sequence set forth in SEQ ID NO: 19, and a second VL comprising the amino acid sequence set forth in SEQ ID NO: 20.

In certain embodiments, the second antigen-binding domain comprises a second VH comprising the amino acid sequence set forth in SEQ ID NO: 28, and a second VL comprising the amino acid sequence set forth in SEQ ID NO: 29.

In certain embodiments, the second antigen-binding domain comprises a second VH comprising the amino acid sequence set forth in SEQ ID NO: 57, and a second VL comprising the amino acid sequence set forth in SEQ ID NO: 58.

In certain embodiments, the first and second antigen-binding domains are selected from the group consisting of Fab, Fab′, (Fab′)₂, Fv, disulfide-linked Fv, scFv, and single domain antibodies (sdAb);

Alternatively, the first antigen-binding domain or the second antigen-binding domain is rabbit derived, murine derived, fully humanized or chimeric.

In certain embodiments, the first antigen-binding domain is Fab.

In certain embodiments, the second antigen-binding domain is scFv, and the second VH of the second antigen-binding domain is linked to the N-terminus or C-terminus of the second VL of the second antigen-binding domain, either directly or by a peptide linker.

In certain embodiments, the peptide linker is (GmS)n, and m and n are independently an integer not less than 0, for example, independently 1, 2, 3, or 4.

In certain embodiments, the peptide linker is GS.

In certain embodiments, the bispecific antibody further comprises an immunoglobulin Fc fragment.

In certain embodiments, the immunoglobulin Fc fragment is linked to the N-terminus and/or C-terminus of the first and second antigen-binding domains, either directly or by a peptide linker.

In certain embodiments, the immunoglobulin Fc fragment is the Fc fragment of a human IgG (eg, IgG1, IgG2, IgG3, or IgG4).

In certain embodiments, the peptide linker is (GmS)n, and m and n are independently an integer not less than 0, for example, independently 1, 2, 3, or 4.

In certain embodiments, the peptide linker is GS.

In certain embodiments, the immunoglobulin Fc fragment comprises a knob or hole mutation.

In certain embodiments, the first antigen-binding domain-linked immunoglobulin Fc fragment comprises a knob mutation and the second antigen-binding domain-linked immunoglobulin Fc fragment comprises a hole mutation.

In certain embodiments, the first antigen-binding domain-linked immunoglobulin Fc fragment comprises a hole mutation and the second antigen-binding domain-linked immunoglobulin Fc fragment comprises a knob mutation.

In certain embodiments, the bispecific antibody is a knob-into-hole format of the bispecific antibody comprising:
(1) peptide chain I-A sequentially comprises the first VL and light chain constant region (CL) of the first antigen-binding domain from the N-terminus to the C-terminus; in certain embodiments, the CL is human immunoglobulin kappa or lambda light chain;
(2) peptide chain I-B sequentially comprises the first VH and heavy chain constant region (CH) of the first antigen-binding domain from the N-terminus to the C-terminus; in certain embodiments, the CH is human immunoglobulin IgG, such as IgG1, IgG2, IgG3 or IgG4;
(3) peptide chain I-C sequentially comprises the second VL of the second antigen-binding domain, the peptide linker, the second VH of the second antigen-binding domain, the peptide linker and the Fc fragment from the N-terminus to the C-terminus; preferably, the peptide linker is (GmS)n, m and n are integers not less than 0, such as 1, 2, 3 or 4; in certain embodiments, the Fc fragment is human IgG (such as IgG1, IgG2, IgG3 or IgG4) of the Fc fragment.

In certain embodiments, the CH3 domain of the Fc fragment of peptide chain I-B comprises a hole mutation, and the CH3 domain of the Fc fragment of peptide chain I-C comprises a knob mutation to promote heterodimerization.

In certain embodiments, the CH3 domain of the Fc fragment of peptide chain I-B comprises mutations T366S, L368A, and Y407V, and the CH3 domain of the Fc fragment of peptide chain I-C comprises mutation T366W.

In certain embodiments, the CH3 domain of the Fc fragment of the peptide chain I-B comprises mutations H435R and Y436F to remove the protein A (Protein A) binding site.

In certain embodiments, the CH1 domain of the Fc fragment of peptide chain I-B comprises mutation N159S to eliminate the deamination reaction.

In certain embodiments, the CH2 domain of the Fc fragment of peptide chains I-B and I-C comprises mutation N297G to reduce ADCC effector function.

In certain embodiments, the peptide chain I-A comprises the sequence set forth in SEQ ID NO: 12, the peptide chain I-B comprises the sequence set forth in SEQ ID NO: 11, and the peptide chain I-C comprises the sequence set forth in any one of SEQ ID NOs: 21, 30 and 59.

In some embodiments, the present invention provides a polypeptide comprising a sequence that is at least 80%, 85%, 90%, 91%, 92%, 93%, 94%, 95%, 96%, 97%, 98%, 99%, or 100% identical to any one of SEQ ID NO: 1-59 as described herein.

### Preparation of Antibodies

The antibodies of the present invention can be prepared by various methods known in the art, such as by genetic engineering and recombinant technology. For example, a DNA molecule encoding the antibody of the present invention is obtained through chemical synthesis or PCR amplification, the obtained DNA molecule is inserted into an expression vector, and then the host cell is transfected. Then, the transfected host cells are cultured under specific conditions and express the antibody of the invention. The antigen-binding fragments of the invention can be obtained by hydrolyzing intact antibody molecules.

In another aspect, the invention provides an isolated nucleic acid molecule encoding a bispecific antibody of the invention.

In another aspect, the invention provides a vector (for example, a cloning vector or an expression vector) comprising an isolated nucleic acid molecule of the invention. In certain embodiments, vectors of the invention are, for example, plasmids, cosmids, phages, and the like.

In another aspect, the invention provides a host cell comprising an isolated nucleic acid molecule or vector as described above. Such host cells include, but are not limited to, prokaryotic cells such as E. coli cells, and eukaryotic cells such as yeast cells, insect cells, plant cells, and animal cells (such as mammalian cells, such as mouse cells, human cells, etc.).

On the other hand, a method for preparing the bispecific antibody of the present invention is provided, comprising the following steps:
the host cells as described above are cultured under conditions that allow protein expression, and the bispecific antibodies are recovered from the cultured host cell culture.

### Conjugate

In another aspect, the invention also provides conjugates comprising a bispecific antibody of the invention and a conjugation moiety.

In certain embodiments, the bispecific antibodies of the invention are optionally conjugated to the coupling moiety via a linker.

In certain embodiments, the coupling moiety is selected from protein tags. Such protein tags are well known in the art, examples of which include but are not limited to His, Flag, GST, MBP, HA, Myc, GFP or biotin, and those skilled in the art know how to select a suitable protein tag according to the desired purpose (e.g., purification tags, detection tags or tracer tags). In certain exemplary embodiments, the bispecific antibodies of the invention have a purification tag attached to their C-terminus.

In certain embodiments, the coupling moiety is selected from a detectable label such as an enzyme (e.g., horseradish peroxidase), a radionuclide, a fluorescent dye, a luminescent substance (e.g., a chemiluminescent substance), or biotin. The detectable label of the present invention can be any substance detectable by fluorescence, spectroscopy, photochemistry, biochemistry, immunology, electrical, optical or chemical means. Such labels are well known in the art and examples include, but are not limited to, enzymes (e.g., horseradish peroxidase, alkaline phosphatase, beta-galactosidase, urease, glucose oxidase, etc.), radionuclides fluorescein (e.g., 3H, 125I, 35S, 14C, or 32P), fluorescent dyes (e.g., fluorescein isothiocyanate (FITC), fluorescein, tetramethylrhodamine isothiocyanate (TRITC), phycoerythrin (PE), Texas Red, rhodamine, quantum dots or cyanine dye derivatives (e.g. Cy7, Alexa 750)), luminescent substances (e.g. chemiluminescent substances such as acridinium esters), magnetic beads (e.g. Dynabeads®), calorimetric labels such as colloidal gold or colored glass or plastic (e.g., polystyrene, polypropylene, latex, etc.) beads, and avidin (e.g., streptavidin) modified to bind the above labels. and biotin). In certain embodiments, such labels can be adapted for immunological detection (e.g., enzyme-linked immunoassay, radioimmunoassay, fluorescent immunoassay, chemiluminescence immunoassay, etc.). In certain embodiments, detectable labels as described above can be linked to bispecific antibodies of the invention via linkers of varying lengths to reduce potential steric hindrance.

In certain embodiments, the coupling moiety is selected from therapeutic agents, such as anti-neoplastic drugs.

In certain embodiments, the coupling moiety is selected from another biologically active polypeptide.

### Pharmaceutical Composition

In one aspect, the invention provides a pharmaceutical composition, comprising the bispecific antibody, isolated nucleic acid molecule, vector, host cell, or conjugate of the invention, and one or more pharmaceutically acceptable Excipients accepted.

In certain embodiments, the pharmaceutical composition may further comprise additional anti-tumor drugs.

In certain embodiments, the bispecific antibody, isolated nucleic acid molecule, vector, host cell, or conjugate of the invention and the additional anti-tumor drug can be used as separate components in the pharmaceutical composition. Available separately or as mixed components. Accordingly, the bispecific antibodies, isolated nucleic acid molecules, vectors, host cells, or conjugates of the invention and the additional anti-tumor agent can be administered simultaneously, separately, or sequentially.

In certain embodiments, the one or more pharmaceutically acceptable excipients can comprise sterile injectable liquids (such as aqueous or non-aqueous suspensions or solutions). In certain exemplary embodiments, such sterile injectable liquid is selected from water for injection (WFI), bacteriostatic water for injection (BWFI), sodium chloride solution (e.g., 0.9% (w/v) NaCl), dextrose Solutions (e.g., 5% glucose), solutions containing surfactants (e.g., 0.01% polysorbate 20), pH buffer solutions (e.g., phosphate buffer solution), Ringer's solution and any combination thereof.

The pharmaceutical composition of the present invention may comprise a “therapeutically effective amount” or a “prophylactically effective amount” of the bispecific antibody, isolated nucleic acid molecule, vector, host cell, or conjugate of the present invention. “Prophylactically effective amount” refers to an amount sufficient to prevent, prevent, or delay the occurrence of disease. A “therapeutically effective amount” means an amount sufficient to cure or at least partially prevent disease and its complications in a patient who is already suffering from the disease. The therapeutically effective amount may vary depending on factors such as the severity of the disease to be treated, the overall state of the patient's own immune system, the patient's general condition such as age, weight and gender, the manner in which the drug is administered, and other concurrent treatments administered, ect.

### Therapeutic Applications

In another aspect, the invention provides a method for preventing and/or treating CD79b-related and/or CD3-related diseases in a subject, comprising administering to a subject in need thereof a bispecific antibody of the invention, isolated nucleic acid molecules, vectors, host cells, conjugates or pharmaceutical compositions of the invention. The invention also relates to the use of said bispecific antibodies, isolated nucleic acid molecules, vectors, host cells, conjugates or pharmaceutical compositions for the preparation of medicaments for prevention and/or treatment of a CD79b-related disease or a CD3-related disease.

In certain embodiments, the CD79b-related disease is characterized by elevated CD79b expression and/or excessive CD79b activity.

In certain embodiments, the CD3-related disease is characterized by elevated CD3 expression and/or excessive CD3 activity.

In certain embodiments, the CD79b-related disease is a B-cell lymphoma-related disease, such as diffuse large B-cell lymphoma (DLBCL), acute B-cell leukemia (B-ALL), chronic lymphocytic leukemia (CLL), B-cell prelymphocytic leukemia (PLL), splenic lymphoma with villous lymphocytes (SLVL), hairy cell leukemia (HCL), follicular lymphoma (FL) and mantle cell lymphoma (MCL).

In certain embodiments, the CD3-related disease is an inflammatory disease or an autoimmune disease.

In certain embodiments, the subject is mammal, such as a human.

In certain embodiments, the bispecific antibody, isolated nucleic acid molecule, vector, host cell, conjugate or pharmaceutical composition is used alone or in combination with an additional anti-tumor agent.

The bispecific antibodies, isolated nucleic acid molecules, vectors, host cells, conjugates or pharmaceutical compositions of the invention can be formulated into any dosage form known in the medical field, for example, tablets, pills, suspensions, Emulsions, solutions, gels, capsules, powders, granules, elixirs, tablets, suppositories, injections (comprising injections, sterile powders for injection and concentrated solutions for injection), inhalants, sprays, etc. The preferred dosage form depends on the intended mode of administration and therapeutic use.

One preferred dosage form is injection. Such injections may be sterile injectable solutions. For example, sterile injectable solutions may be prepared by incorporating in an appropriate solvent the requisite dose of an antibody of the invention, or antigen-binding fragment thereof, and, optionally, other desired ingredients comprising, but not (limited to, pH adjuster, surfactant, adjuvant, ionic strength enhancer, isotonic agent, preservative, diluent, or any combination thereof), followed by filter sterilization. Additionally, sterile injectable solutions may be prepared as sterile lyophilized powders (for example, by vacuum drying or freeze drying) for ease of storage and use. Such sterile lyophilized powder can be dispersed in a suitable carrier before use, such as water for injection (WFI), bacteriostatic water for injection (BWFI), sodium chloride solution (such as 0.9% (w/v) NaCl), Glucose solution (e.g. 5% glucose), surfactant-containing solution (e.g. 0.01% polysorbate 20), pH buffer solution (e.g. phosphate buffer solution), Ringer's solution and any combination thereof.

The bispecific antibodies, isolated nucleic acid molecules, vectors, host cells, conjugates or pharmaceutical compositions of the invention may be administered by any suitable method known in the art, comprising, but not limited to, oral, buccal, sublingual, intraocular, topical, parenteral, rectal, intrathecal, intracytoplasmic reticulum, inguinal, intravesical, topical (e.g., powder, ointment, or drops), or nasal route. However, for many therapeutic uses, the preferred route/mode of administration is parenteral (e.g., intravenous or bolus injection, subcutaneous injection, intraperitoneal injection, intramuscular injection). The skilled artisan will understand that the route and/or mode of administration will vary depending on the intended purpose. In certain embodiments, the bispecific antibodies, isolated nucleic acid molecules, vectors, host cells, or conjugates of the invention or pharmaceutical compositions of the invention are administered by intravenous injection or bolus injection.

### Detection Application

In another aspect, the invention provides a method of detecting the presence or content of CD79b and/or CD3 in a sample, comprising the use of a bispecific antibody or conjugate of the invention.

In certain embodiments, the method is an immunological assay, such as a Western blot, an enzyme immunoassay (e.g. ELISA), a chemiluminescent immunoassay, a fluorescent immunoassay, or a radioimmunoassay.

In certain embodiments, conjugates for use in the methods comprise a bispecific antibody of the invention and a detectable label.

In certain embodiments, bispecific antibodies used in the methods are detectably labeled.

In certain embodiments, bispecific antibodies used in the methods do not bear a detectable label. Accordingly, the method may also comprise detecting the bispecific antibodies of the invention using other reagents (e.g. secondary antibodies) with a detectable label.

In certain embodiments, the method comprises the steps of
(1) contact the sample with the bispecific antibody or conjugate of the invention;
(2) the formation of a complex between the bispecific antibody or conjugate and the antigen or the amount of the complex is detected.

The formation of the complex indicates the presence of the antigen or cells expressing the antigen;
wherein, the antigen is selected from CD79b or CD3.

The method may be used for diagnostic purposes, or for non-diagnostic purposes (for example, the sample is a cell sample rather than a sample from a patient).

In certain embodiments, the methods are used to diagnose whether a subject has a CD79b-related and/or CD3-related disease. In such embodiments, the method may further comprise the step of comparing the amount of CD79b and/or CD3 in a sample from the subject to a reference value. The reference value may be the level of CD79b and/or CD3 in a sample from a subject known not to have a CD79b-related and/or CD3-related disease (e.g. a healthy control) (also referred to a “negative reference value”). For example, if the amount of CD79b and/or CD3 in a sample from the subject is elevated relative to a negative reference value, it is an indication that the subject suffers from a CD79b-related and/or CD3-related disease.

In certain embodiments, the CD79b-related disease is characterized by elevated CD79b expression and/or excessive CD79b activity. In certain embodiments, the CD79b-related disease is a B-cell lymphoma-related disease, such as diffuse large B-cell lymphoma (DLBCL), acute B-cell leukemia (B-ALL), chronic lymphocytic leukemia (CLL), B-cell prelymphocytic leukemia (PLL), splenic lymphoma with villous lymphocytes (SLVL), hairy cell leukemia (HCL), follicular lymphoma (FL) and mantle cell lymphoma (MCL).

In certain embodiments, the CD3-related disease is characterized by elevated CD3 expression and/or excessive CD3 activity. In certain embodiments, the CD3-related disease is an inflammatory disease or an autoimmune disease.

In certain embodiments, the sample may be selected from urine, blood, serum, plasma, saliva, ascites fluid, circulating cells, circulating tumor cells, non-tissue associated cells (i.e., free cells), tissue (e.g., surgically resected tumor tissue, biopsy or fine needle aspiration tissue), histological preparations, etc.

In certain embodiments, the CD79b is human CD79b.

In certain embodiments, the CD3 is human CD3.

On the other hand, there is provided the use of the bispecific antibody or conjugate of the present invention in the preparation of detection reagents for detecting the presence or levels of CD79b and/or CD3 in a sample or for diagnosing affected Whether the subject has CD79b-related and/or CD3-related diseases.

In certain embodiments, conjugates used to prepare detection reagents comprise a bispecific antibody of the invention and a detectable label.

In certain embodiments, bispecific antibodies used to prepare detection reagents are detectably labeled.

In certain embodiments, the bispecific antibodies used to prepare detection reagents do not bear a detectable label. In such embodiments, the detection reagents may further comprise other reagents (e.g. secondary antibodies) capable of detecting the bispecific antibodies of the invention.

### Definition of Terms

In the present invention, unless otherwise stated, scientific and technical terms used herein have the meanings commonly understood by those skilled in the art. Moreover, the cell culture, molecular biology, biochemistry, nucleic acid chemistry, immunology and other operating procedures used in this article are routine procedures widely used in the corresponding fields. Meanwhile, to better understand the present invention, definitions and explanations of relevant terms are provided below.

When the terms “for example” “such as” “e.g.” “comprising” “comprises” or variant thereof are used herein, these terms will not be considered limiting terms and will instead be interpreted to mean “but without limitation” or “without limitation”

Unless otherwise indicated herein or clearly contradicted by context, the terms “a” and “an” as well as “the” and similar referents in the context of describing the invention (especially in the context of the following claims) are to be construed to cover singular and plural.

As used herein, the term “CD79b” refers to a single-stranded membrane protein that constitutes CD79 and contains an extracellular domain, a transmembrane domain, and an intracellular domain. The extracellular domain interacts with the extracellular domain of the Igα chain to form the external structure of the B cell receptor complex. The intracellular domain participates in signaling pathways and regulates the biological functions of B cells. The sequence of CD79b is well known to those skilled in the art (see, e.g., NCBI Protein database accession number: AAH32651).

As used herein, the term “CD3” refers to a protein complex having four chains. In mammals, the complex contains one CD3 γ (γ) chain, one CD3 δ (δ) chain, and two CD3e (ε) chains. These chains bind to the T cell receptor (TCR) and the so-called ζ (ζ) chain to form the T cell receptor CD3 complex and generate an activation signal in T lymphocytes. The sequence of CD3e is well known to those skilled in the art (see, e.g., NCBI Protein database accession number: NP_000724).

As used herein, the term “antibody” in its broadest sense refers to molecules that specifically bind to an antigenic determinant and may comprise a variety of antibody structures so long as they exhibit the desired antigen-binding activity. Typically, a wildtype antibody may be an immunoglobulin molecule consisting of two pairs of polypeptide chains, each pair having a light chain (LC) and a heavy chain (HC). Antibody light chains can be classified into kappa (kappa) and lambda (lambda) light chains. Heavy chains can be classified as mu, delta, gamma, alpha, or epsilon, and define the antibody's isotype as IgM, IgD, IgG, IgA, and IgE, respectively. Within the light and heavy chains, the variable and constant regions are connected by a “J” region of approximately 12 or more amino acids, and the heavy chain also contains a “D” region of approximately 3 or more amino acids. Each heavy chain has a heavy chain variable region (VH) and a heavy chain constant region (CH). The heavy chain constant region has 3 domains (CH1, CH2 and CH3). Each light chain has a light chain variable region (VL) and a light chain constant region (CL). The light chain constant region has one domain, CL. The constant domain is not directly involved in the binding of antibodies to antigens, but exhibits a variety of effector functions, such as mediating the interaction of immunoglobulins with host tissues or factors, comprising various cells of the immune system (e.g., effector cells) and classical complement. Binding of the first component of the system (C1q). The VH and VL regions can also be subdivided into regions of high variability called complementarity determining regions (CDRs), interspersed with more conservative regions called framework regions (FRs). Each VH and VL has 3 CDRs and 4 FRs arranged from the amino terminus to the carboxyl terminus in the following order: FR1, CDR1, FR2, CDR2, FR3, CDR3, FR4. The variable regions (VH and VL) of each heavy chain/light chain pair respectively form the antigen-binding site. The assignment of amino acids to regions or domains can follow Kabat, Sequences of Proteins of Immunological Interest (National Institutes of Health, Bethesda, Md. (1987 and 1991)), or Chothia & Lesk (1987) J. Mol. Biol. 196:901-917; Definition of Chothia et al. (1989) Nature 342:878-883.

As used herein, the term “bispecific antibody” refers to an antibody that has binding specificities for two different antigens (or epitopes). Bispecific antibodies comprise two antigen-binding domains with binding specificities for different antigens (or epitopes), thereby being able to bind to two different binding sites and/or target molecules.

As used herein, the term “Fab fragment” refers to a fragment of an antibody, wherein the fragment comprises a heavy chain variable domain, a light chain variable domain, and a light chain constant domain. and the first constant domain (CH1) of the heavy chain.

As used herein, the term “single chain variable fragment (scFv)” is a fusion protein of the heavy chain variable region (VH) and the light chain variable region (VL), optionally with 10 to about 25 amino acids short linker peptide. VH is linked to the N-terminus or C-terminus of VL by a short peptide. The scFv does not comprise a constant region but retains the specificity of the original antibody.

As used herein, the term “complementarity determining region” or “CDR” refers to the amino acid residues in the variable region of an antibody that are responsible for antigen binding. The variable regions of the heavy chain and light chain each contain three CDRs, named CDR1, CDR2 and CDR3. The precise boundaries of these CDRs can be defined according to various numbering systems known in the art, such as the Kabat numbering system (Kabat et al., Sequences of Proteins of Immunological Interest, 5th Ed. Public Health Service, National Institutes of Health, Bethesda, Md., 1991), the Chothia numbering system (Chothia & Lesk (1987) J. Mol. Biol. 196:901-917; Chothia et al. (1989) Nature 342:878-883) or the IMGT numbering system (Lefranc et al. al., Dev. Comparat. Immunol. 27:55-77, 2003). For a given antibody, one skilled in the art will readily identify the CDRs defined by each numbering system. Moreover, the correspondence between different numbering systems is well known to those skilled in the art (for example, see Lefranc et al., Dev. Comparat. Immunol. 27:55-77, 2003).

As used herein, the term “framework region” or “FR” residues refers to those amino acid residues in an antibody variable region other than the CDR residues as defined above.

As used herein, the term “knob-into-hole” is a bispecific antibody development technique that involves the introduction of “knobs” (protrusions) into a first polypeptide and into a second polypeptide. The corresponding “hole” allows the “knob” to be positioned in the “hole” to promote the formation of heterodimers and hinder the formation of homodimers. “knobs” are constructed by replacing small amino acid side chains from the first polypeptide with larger side chains, such as tyrosine or tryptophan. A compensating “hole” of the same or similar size as the “knob” is created in the interface of the second polypeptide by replacing a large amino acid side chain with a smaller amino acid side chain (such as alanine or threonine). “knobs” and “holes” can be prepared by altering the nucleic acid encoding the polypeptide, for example by site-specific mutagenesis or by peptide synthesis.

As used herein, the term “specific binding” refers to a non-random binding reaction between two molecules, such as the reaction between an antibody and the antigen against which it is directed. The strength or affinity of a specific binding interaction can be expressed by the equilibrium dissociation constant (KD) of the interaction. In the present invention, the term “KD” refers to the dissociation equilibrium constant of a specific antibody-antigen interaction, which is used to describe the binding affinity between an antibody and an antigen. The smaller the equilibrium dissociation constant, the tighter the antibody-antigen binding, and the higher the affinity between the antibody and the antigen.

The specific binding properties between two molecules can be determined using methods known in the art. One approach involves measuring the rate at which antigen binding site/antigen complexes form and dissociate. Both the “association rate constant” (ka or kon) and the “dissociation rate constant” (kdis or koff) can be calculated from the concentration and the actual rates of association and dissociation (see Malmqvist M, Nature, 1993, 361:186-187). The ratio of kdis/kon is equal to the dissociation constant KD (see Davies et al., Annual Rev Biochem, 1990; 59:439-473). KD, kon and kdis values can be measured by any valid method. In certain embodiments, dissociation constants can be measured in Biacore using surface plasmon resonance (SPR). Alternatively, bioluminescence interferometry or Kinexa can be used to measure dissociation constants.

As used herein, the term “vector” refers to a nucleic acid delivery vehicle into which a polynucleotide can be inserted. When the vector can express the protein encoded by the inserted polynucleotide, the vector is called an expression vector. The vector can be introduced into the host cell through transformation, transduction or transfection, so that the genetic material elements it carries can be expressed in the host cell. Vectors are well known to those skilled in the art, comprising but not limited to plasmids; phagemids; cosmids; artificial chromosomes, such as yeast artificial chromosomes (YAC), bacterial artificial chromosomes (BAC) or P1-derived artificial chromosomes (PAC); Phages such as lambda phage or M13 phage and animal viruses, etc. Animal viruses that can be used as vectors include, but are not limited to, retroviruses (comprising lentiviruses), adenoviruses, adeno-associated viruses, herpesviruses (such as herpes simplex virus), poxviruses, baculoviruses, papillomaviruses, papillomaviruses, Polyomavacuolating viruses (such as SV40). A vector can contain a variety of expression-controlling elements, comprising, but not limited to, promoter sequences, transcription initiation sequences, enhancer sequences, selection elements, and reporter genes. In addition, the vector may also contain an origin of replication site.

As used herein, the term “host cell” refers to a cell that can be used to introduce a vector, which includes, but is not limited to, prokaryotic cells such as E. coli or Bacillus subtilis, fungal cells such as yeast cells or Aspergillus, etc., insect cells such as S2 Drosophila cells or Sf9, or animal cells such as fibroblasts, CHO cells, COS cells, NSO cells, HeLa cells, BHK cells, HEK 293 cells or human cells. Host cells can include single cells or populations of cells.

As used herein, the term “identity” is used to refer to the match of sequences between two polypeptides or between two nucleic acids. When a position in both sequences being compared is occupied by the same base or amino acid monomer subunit (for example, a position in each of two DNA molecules is occupied by adenine, or two A certain position in each polypeptide is occupied by lysine), then the molecules are identical at that position. “Percent identity” between two sequences is a function of the number of matching positions common to the two sequences divided by the number of positions compared×100. For example, if 6 out of 10 positions of two sequences match, then the two sequences are 60% identical. For example, the DNA sequences CTGACT and CAGGTT share a 50% identity (matching at 3 out of 6 total positions). Typically, comparisons are made when two sequences are aligned to yield maximum identity. Such alignment can be accomplished using, for example, the method of Needleman et al. (1970) J. Mol. Biol. 48:443-453, which can be conveniently performed by a computer program such as the Align program (DNAstar, Inc.). It is also possible to use the PAM120 weight residue table using the algorithm of E. Meyers and W. Miller (Comput. Appl Biosci., 4:11-17 (1988)) integrated into the ALIGN program (version 2.0), a gap length penalty of 12 and a gap penalty of 4 to determine the percent identity between two amino acid sequences. Alternatively, the Needleman and Wunsch (J MoI Biol. 48:444-453 (1970)) algorithm can be used using the Blossum 62 matrix or PAM250 matrix with a gap weight of 16, 14, 12, 10, 8, 6 or 4 and a length weight of 1, 2, 3, 4, 5 or 6 to determine the percent identity between two amino acid sequences.

As used herein, the term “conservative substitution” means an amino acid substitution that does not adversely affect or alter the expected properties of the protein/polypeptide comprising the amino acid sequence. For example, conservative substitutions can be introduced by standard techniques known in the art, such as site-directed mutagenesis and PCR-mediated mutagenesis. Conservative amino acid substitutions include those in which an amino acid residue is replaced with an amino acid residue having a similar side chain, e.g., one that is physically or functionally similar to the corresponding amino acid residue (e.g., has similar size, shape, charge, chemical properties, comprising ability to form covalent bonds or hydrogen bonds, etc.). Families of amino acid residues with similar side chains have been defined in the art. These families include those with basic side chains (e.g., lysine, arginine, and histidine), acidic side chains (e.g., aspartic acid, glutamic acid), uncharged polar side chains (e.g., glycine asparagine, glutamine, serine, threonine, tyrosine, cysteine, tryptophan), non-polar side chains (such as alanine, valine, leucine, isoleucine amino acids, proline, phenylalanine, methionine), β-branched side chains (e.g., threonine, valine, isoleucine) and aromatic side chains (e.g., tyrosine, Phenylalanine, tryptophan, histidine) amino acids. Therefore, it is preferred to replace the corresponding amino acid residue with another amino acid residue from the same side chain family. Methods for identifying conservative substitutions of amino acids are well known in the art (see, e.g., Brummell et al., Biochem. 32:1180-1187 (1993); Kobayashi et al., Protein Eng. 12(10):879-884 (1999); and Burks et al. Proc. Natl Acad. Set USA 94:412-417 (1997), which is incorporated herein by reference).

The twenty conventional amino acids involved in this article have been prepared following conventional usage. See, e.g., Immunology-A Synthesis (2nd Edition, E. S. Golub and D. R. Gren, Eds., Sinauer Associates, Sunderland, Mass. (1991)), which is incorporated herein by reference. In the present invention, the terms “polypeptide” and “protein” have the same meaning and are used interchangeably. And in the present invention, amino acids are generally represented by one-letter and three-letter abbreviations well known in the art. For example, alanine can be represented by A or Ala.

As used herein, the term “pharmaceutically acceptable excipient” refers to excipients that are pharmacologically and/or physiologically compatible with the subject and the active ingredient and are well known in the art (see, e.g., Remington's Pharmaceutical Sciences. Edited by Gennaro AR, 19th ed. Pennsylvania: Mack Publishing Company, 1995), and include, but are not limited to: pH adjusters, surfactants, adjuvants, ionic strength enhancers, diluents, agents to maintain osmotic pressure, delays Absorbed reagents, preservatives. For example, pH adjusting agents include, but are not limited to, phosphate buffer. Surfactants include, but are not limited to, cationic, anionic or nonionic surfactants such as Tween-80. Ionic strength enhancers include, but are not limited to, sodium chloride. Preservatives include, but are not limited to, various antibacterial and antifungal agents, such as parabens, chlorobutanol, phenol, sorbic acid, etc. Agents that maintain osmotic pressure include, but are not limited to, sugar, NaCl, and the like. Agents that delay absorption include, but are not limited to, monostearate and gelatin. Diluents include, but are not limited to, water, aqueous buffers (such as buffered saline), alcohols and polyols (such as glycerol), and the like.

As used herein, the term “prevention” refers to a method performed to prevent or delay the occurrence of a disease or condition or symptom (e.g., a disease associated with CD79b and/or CD3) in a subject. As used herein, the term “treatment” refers to a method performed to obtain a beneficial or desired clinical result. For the purposes of this invention, beneficial or desired clinical results include, but are not limited to, alleviation of symptoms, reduction of the extent of the disease, stabilization (i.e., no worsening) of the disease state, delaying or slowing the progression of the disease, ameliorating or alleviating the symptoms of the disease. status, and relief of symptoms (whether partial or complete), whether detectable or undetectable. In addition, “treatment” may also refer to prolonging survival compared with expected survival if no treatment was received.

As used herein, the term “subject” refers to mammal, such as primate mammal, such as humanized. In certain embodiments, the subject (e.g., human) has a disease associated with CD79b. In certain embodiments, the subject (e.g., human) has a disease associated with CD3.

In the present invention, unless otherwise specified, the “first” (for example, the first antigen or the first antigen-binding domain) and the “second” (for example, the second antigen or the second antigen-binding domain) are mainly for the purpose of referring to the generational distinction does not have a typical sequential meaning.

### Beneficial Effects of the Invention

The present invention provides bispecific antibodies that can simultaneously target CD79b and CD3. On the basis of treating CD79b-related diseases (such as B-cell lymphoma), they further mediate the killing of tumor cells by T cells and are compatible with single-specific antibodies. Compared with sexual antibodies, it has higher therapeutic potential.

The embodiments of the present invention will be described in detail below with reference to the accompanying drawings and examples, but those skilled in the art will understand that the following drawings and examples are only used to illustrate the present invention and do not limit the scope of the present invention. The various objects and advantageous aspects of the present invention will become apparent to those skilled in the art from the accompanying drawings and the following detailed description of preferred embodiments.

### DESCRIPTION OF THE DRAWINGS

FIG. 1: Schematic diagram of antibody structure.

FIG. 2: Binding of different CD79b/CD3 bispecific antibodies to CD79b.

FIG. 3: Binding of different CD79b/CD3 bispecific antibodies to CD3.

FIG. 4: Binding of different CD79b/CD3 bispecific antibodies to Jurkat cells.

FIG. 5: Activation of T cells by different CD79b/CD3 bispecific antibodies.

FIG. 6: CD8 T cell mediated cancer cell killing induced by different CD79b/CD3 bispecific antibodies.

FIG. 7: Changes in granzyme B when different CD79b/CD3 bispecific antibodies were used to activate CD8 T cells to perform their cell killing functions.

FIG. 8: Ki67 changes when different CD79b/CD3 bispecific antibodies were used to activate CD8 T cells to perform their cell killing functions.

FIG. 9: Cytokine TNFα release induced by different CD79b/CD3 bispecific antibodies.

FIG. 10: Cytokine IFNγ release induced by different CD79b/CD3 bispecific antibodies.

FIGS. 11A-11E: relevant amino acid sequence discussed herein.

### EXAMPLES

The invention is further described in the following examples, which do not limit the scope of the invention described in the claims.

Those skilled in the art will appreciate that the examples describe the invention by way of example and are not intended to limit the scope of protection claimed in the application. The experimental methods in the examples are all conventional methods unless otherwise specified. If the specific conditions are not specified in the examples, the conditions should be carried out according to the conventional conditions or the conditions recommended by the manufacturer. If the manufacturer of the reagents or instruments used is not indicated, they are all conventional products that can be purchased commercially.

### Example 1: Molecular Construction

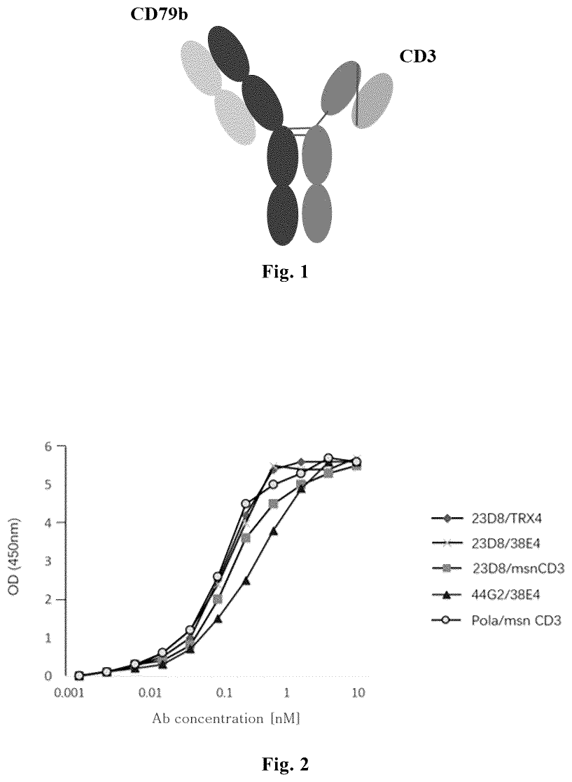

Bispecific antibodies were constructed based on the structure shown in FIG. 1.

To express chimeric rabbit/human IgG1 antibodies, constant regions of the heavy chain (CH1-3) of human IgG1 (SEQ ID NO: 1) and the constant region of human light chain kappa (CL-κ) (SEQ ID NO: 2) were synthesized and cloned into pcDNA3.4 respectively (GeneScript). pcDNA3.4 containing CH1-3 of human IgG1 (pcDNA3.4-huIgG1-Hc) was further digested with EcoRI/Nhel for cloning of VH sequence. And EcoRI/BsiWI were used to digest VL sequences receiving vector, pcDNA3.4 expressing human CL-κ (pcDNA3.4-huKappa-Lc). VH (SEQ ID NOs: 9, 37 and 47) and VL sequences (SEQ ID NOs: 10, 38 and 48) of selected rabbit anti-CD79b antibodies (23D8 and 44G2) and that of the reference antibody Polatuzumab, were synthesized (IDT) with designed overlapped sequences at both 5′ and 3′ ends which will anneal and assemble (NEB NEBuilder® HiFi DNA Assembly) with the corresponding ends of receiving vectors.

To generate a CD79b/CD3 bispecific antibody, the scFv (single-chain fragment variable VL-GS-VH) of CD3 antibody was connected to the shortened Fc (CH2-3) knob meanwhile the CD79b VH was linked with Fc Hole. Fragments of Hole Fc, Knob Fc and scFv of CD3 binding portion of Mosunetuzumab (hereinafter referred to as MsnCD3), 38E4 and TRX4 (otelixizumab) were synthesized (IDT) and assembled (NEB NEBuilder® HiFi DNA Assembly) into pcDNA3.4-huIgG1-Hc or pcDNA3.4-huKappa-Lc carrying desired fragments as described above. The partial composition and sequence of the CD79b antibody are shown in Table 1 below. The partial composition and sequence of the CD3 antibody are shown in Table 2 below. The specific composition and sequence of the CD79b/CD3 bispecific antibody are shown in Table 3 below.

TABLE 1
Partial composition and sequence of CD79b antibody

| CD79b | VH | VL |
|---|---|---|
| antibody | (SEQ ID NO:) | (SEQ ID NO:) |
| 23D8 | 9 | 10 |
| 44G2 | 37 | 38 |
| Pola | 47 | 48 |

TABLE 2
Partial composition and sequence of CD3 antibodies

| CD3 | VH | VL |
|---|---|---|
| antibody | (SEQ ID NO:) | (SEQ ID NO:) |
| msnCD3 | 57 | 58 |
| TRX4 | 19 | 20 |
| 38E4 | 28 | 29 |

TABLE 3
Composition and sequence of CD79b/CD3 bispecific antibodies

| | Peptide I-A (anti- | Peptide I-B (anti- | |
|---|---|---|---|
| | CD79b | CD79b | Peptide I-C (anti- |
| CD79b/CD3 | antibody light | antibody heavy | CD3 |
| bispecific | chain) | chain) | scFv-Fc) |
| antibody | SEQ ID NO: | SEQ ID NO: | SEQ ID NO: |
| 23D8/TRX4 | 12 | 11 | 21 |
| 23D8/38E4 | 12 | 11 | 30 |
| 23D8/msnCD3 | 12 | 11 | 59 |
| 44G2/38E4 | 40 | 39 | 31 |
| Pola/msnCD3 | 50 | 49 | 59 |

The assembled plasmids were used to transform Competent E. coli (NEB® 5-alpha) cells, and clones with correct sequences, based on sequencing results (Elim Biopharm), were further cultured with LB containing Carbenicillin (100 μg/ml) for plasmid purification (QIAGEN Plasmid Plus Kits). Plasmids were eluted in Nuclease-free H₂O (Sigma) and stored at −80° C.

### Example 2: Transfection and Purification of Antibodies

Antibodies were expressed with CHO cells (ExpiCHO™ Expression System, Gibco) by transfection of both pcDNA3.4-huIgG1-Hc and pcDNA3.4-huKappa-Lc containing paired VH and VL sequence. ExpiCHO cells were cultured with ExpiCHO expression medium and maintained between 0.3 to 6×10⁶/ml following manufacturer recommendation at 37° C., 125 rpm, 5% CO₂ and 80% humidity. In a 125 ml baffled flask, 25 ml of fresh ExpiCHO cells (6×10⁶/ml and viability>95%) were prepared from 1 day-long culture seeded at 3×10⁶/ml. 1 ml serum-free medium (OptiPRO™ SFM, Gibco) containing both pcDNA3.4-huIgG1-Hc and pcDNA3.4-huKappa-Lc (12 ug each plasmid) was mixed well by pipetting with 1 ml OptiPRO™ SFM containing 80 ul transfection reagent (ExpiFectamine™ CHO Reagent, Gibco). The transfection mixture was then added to the 25 ml ExpiCHO cells and cultured at 37° C. On the next day, the transfection culture was transferred to 32° C. incubator after adding of 150 ul ExpiFectamine™ CHO Enhancer, 6 ml ExpiCHO™ Feed and 1× Penicillin-Streptomycin (Gibco). The cell density and viability of the transfection culture were monitored and the IgG1 antibody titer in the medium was determined using BLI technology with Protein A biosensor (GatorBio). Around 5 days later, the culture medium containing secreted IgG1 antibodies were collected (2000 g, 10 minutes), filtered (Thermo Scientific™ Nalgene™ Rapid-Flow™ Sterile Disposable Filter) and further purified using Protein A resin (TOYOPEARL AF-rProtein A He-650F) packed gravity-flow column (Bio-Rad). IgG1 antibodies were eluted with 3.5 ml Glycine-HCl (100 mM, pH 2.7), immediately neutralized with 1M Tris-HCl (pH8.5), dialyzed with Thermo Scientific™ Slide-A-Lyzer™ G2 Dialysis Cassettes (20K MWCO) in 1×PBS buffer (pH 7.2) and stored at 4° C. The concentration of purified IgG1 antibody was determined with NanoDrop™ One/OneC Microvolume UV-Vis Spectrophotometer (Thermo Scientific™) and the quality of IgG1 antibody was checked by SDS-PAGE gel under both denaturing and non-denaturing conditions. Five CD79b/CD3 bispecific antibodies, 23D8/TRX4, 23D8/38E4, 23D8/msnCD3, 44G2/38E4 and Pola/msnCD3, were obtained and used in the following examples.

### Example 3: CD79b/CD3 Bispecific Antibody Binds to CD79b

The binding affinities of CD79b/CD3 bispecific antibodies to human CD79b recombinant protein were tested by ELISA. 1 μg/ml (in PBS buffer) of human CD79b recombinant protein was coated on ELISA plates overnight. ELISA plates were washed and blocked with blocking buffer (PBS+1% BSA) and then incubated with serial dilutions of primary CD79b/CD3 bispecific antibodies. Binding to CD79b was quantified using an anti-human IgG HRP secondary antibody (Biolegend catalog number 410902) and the HRP substrate. The binding affinities (as determined by ELISA) of various bispecific antibodies to human CD79b recombinant protein are shown in FIG. 2. The results showed that 23D8/TRX4, 23D8/38E4, 23D8/msnCD3, 44G2/38E4 and Pola/msnCD3 could bind to CD79b.

### Example 4: CD79b/CD3 Bispecific Antibody Binds to CD3

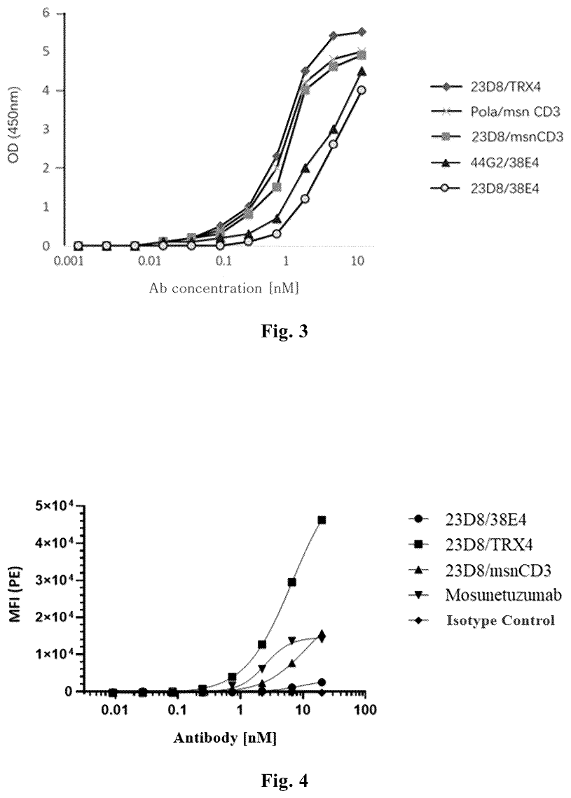

The binding affinities of CD79b/CD3 bispecific antibodies to human CD3 recombinant protein were tested by ELISA. 1 μg/ml (in PBS buffer) of human CD3 recombinant protein was coated on ELISA plates overnight. ELISA plates were washed and blocked with blocking buffer (PBS+1% BSA) and then incubated with serial dilutions of primary CD79b/CD3 bispecific antibodies. Binding to CD3 was quantified using an anti-human IgG HRP secondary antibody (Biolegend catalog number 410902) and the HRP substrate. The binding affinities (as measured by ELISA) of various bispecific antibodies to human CD3 recombinant protein are shown in FIG. 3. The results showed that the CD79b/CD3 bispecific antibodies 23D8/TRX4, 23D8/38E4, 23D8/msnCD3, 44G2/38E4 and Pola/msnCD3 could bind to CD3.

### Example 5: CD79b/CD3 Bispecific Antibody Binds to Jurkat Cells

To compare the binding affinities of different CD3 binders in CD79b/CD3 bispecific antibodies, Jurkat cells were used as a target cells. Jurkat cells were washed twice with FACS buffer (1×PBS+2% BSA+2 mM EDTA). 5×10⁴ target cells in 50 μl FACS buffer were seeded in each well of a V bottom 96-well plate. Then, 50 μl of serially diluted CD79b/CD3 bispecific antibodies 23D8/38E4, 23D8/TRX4, 23D8/msnCD3, Mosunetuzumab, and the negative control Human IgG Isotype Control (Invitrogen Catalog #02-7102) were separately added into different wells. The mixture was incubated on ice for 30 minutes; then, the cells were washed twice with FACS buffer (centrifuged at 1200 rpm for 5 minutes). The cells were resuspended in 100 μl FACS buffer containing PE conjugated goat anti-human IgG Fc (1:1000)(Invitrogen), incubated for 30 minutes on ice in darkness. After removing the staining buffer and washing twice with FACS buffer, cells were resuspended in 100 μl FACS buffer and analyzed with Cytek cytometer. The results are shown in Table 4 and FIG. 4. The bispecific antibody 23D8/TRX4 showed a better binding affinity than other bispecific antibodies.

TABLE 4
Binding of CD79b/CD3 bispecific antibodies to Jurkat cells

| Antibody | IC50 |
|---|---|
| 23D8/38E4 | 9.393 |
| 23D8/TRX4 | 6.624 |
| 23D8/msnCD3 | 11.35 |
| Mosunetuzumab | 2.483 |

### Example 6: Activation of T Cells by CD79b/CD3 Bispecific Antibodies

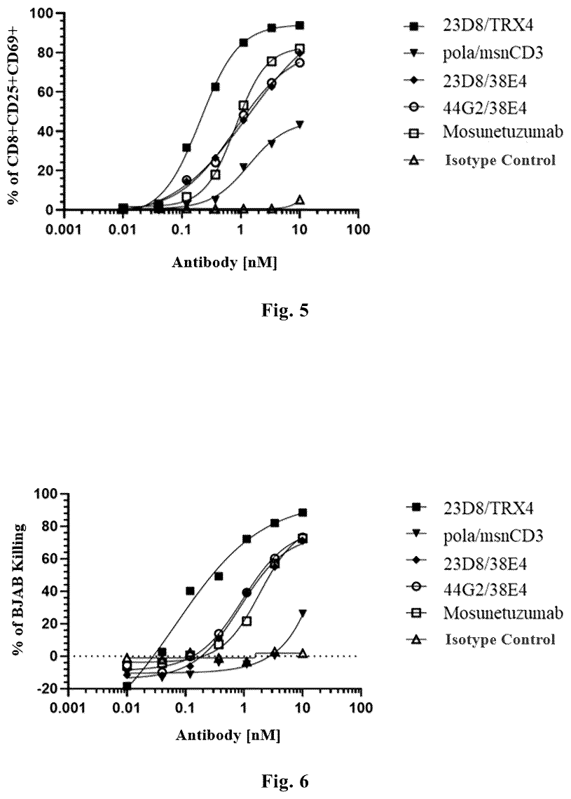

To test the ability of different CD79b/CD3 bispecific antibodies to induce CD8 T cell activation, a coculture system of human CD8 T cells and BJAB target cells was set up. CD8 T cells were isolated and enriched from human PBMC with StemCell Kit. Frozen CD8 T cells were thawed and washed one time with 10 ml fresh medium. 15 μl of CD79b/CD3 bispecific antibodies 23D8/TRX4, pola/msnCD3, 23D8/38E4, 44G2/38E4, Mosunetuzumab, and negative control Human IgG Isotype Control (Invitrogen Catalog #02-7102) serially diluted in PBS were separately added into different wells of a U bottom 96-well plate. 1×10⁵ CD8 T cells and 2×10⁴ BJAB target cells (5:1 ratio) in 135 μl complete culture medium (RPMI1640+10% FBS) were added into each well respectively. The mixture was incubated at 37° C.+5% CO₂ for 48 hours. Cells were centrifuged at 1500 rpm for 5 minutes. Cell pellets were resuspended in FACS buffer, stained and analyzed with Cytek cytometer. The staining panels included live/dead dye syntox blue, PEcy7 CD8, AF700 CD4, APC CD25, and APCcy7 CD69, and L/D-stained CD8+CD25+CD69+ cells were gated as active CD8 T cells. The results are shown in FIG. 5. 23D8/TRX4 showed the best ability to activate CD8 T cells.

### Example 7: CD79b/CD3 Bispecific Antibody Kills Tumor Cells Mediated by T Cells

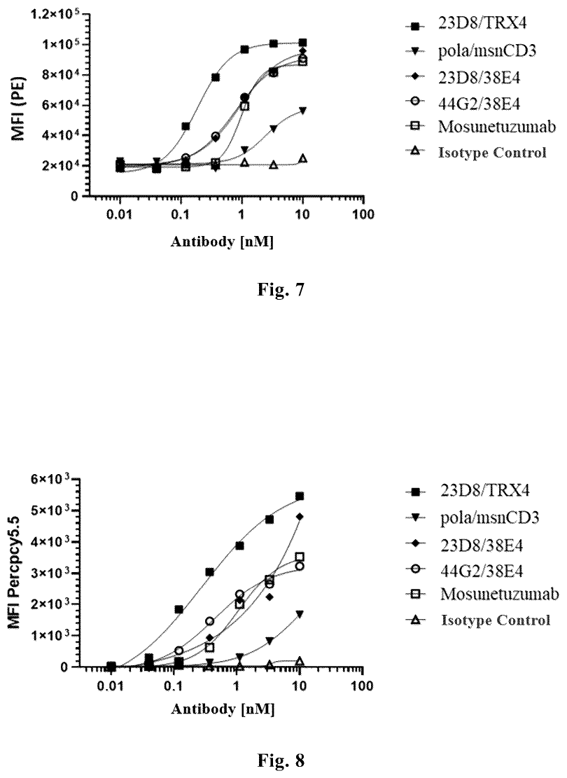

To test the efficacy of different CD79b/CD3 bispecific antibodies in killing target tumor cells, a coculture system of human CD8 T cells with BJAB target cells was set up. CD8 T cells were isolated and enriched from human PBMC with StemCell Kit. BJAB cells were stained with CFSE (Invitrogen). 15 μl of CD79b/CD3 bispecific antibodies 23D8/TRX4, pola/msnCD3, 23D8/38E4, 44G2/38E4, Mosunetuzumab, and negative control Human IgG Isotype Control (Invitrogen Catalog #02-7102) serially diluted in PBS were separately added into different wells of a U bottom 96-well plate. 1×10⁵ CD8 T cells and 2×10⁴ BJAB target cells (5:1 ratio) in 135 μl complete culture medium (RPMI1640+10% FBS) were added into each well respectively. The mixture was incubated at 37° C.+5% CO₂ for 48 hours. Cells were centrifuged at 1500 rpm for 5 minutes. Cell pellets were resuspended in FACS buffer, stained and analyzed with Cytek cytometer. The staining panel includes live/dead dye syntox blue, BV421 CD19. L/D CFSE+ cells were gated as surviving tumor cells. The killing efficacy was calculated by the formula: killing=(number of control tumor cells−number of control tumor cells)/number of control tumor cells. The results are shown in FIG. 6. 23D8/TRX4 showed the best killing efficacy among the candidates tested.

### Example 8: Cytokine Release from CD79b/CD3 Bispecific Antibodies

To understand the changes in granzyme B and Ki67 in CD8 T cells while activated and executing killing function, a coculture system of human CD8 T cells with BJAB target cells was set up. CD8 T cells were isolated and enriched from human PBMC with StemCell kit. BJAB cells were stained with CFSE (Invitrogen). 15 μl of CD79b/CD3 bispecific antibodies 23D8/TRX4, pola/msnCD3, 23D8/38E4, 44G2/38E4, Mosunetuzumab, and negative control Human IgG Isotype Control (Invitrogen Catalog #02-7102) serially diluted in PBS were separately added into different wells of a U bottom 96-well plate. 1×10⁵ CD8 T cells and 2×10⁴ BJAB target cells (5:1 ratio) in 135 μl complete culture medium (RPMI1640+10% FBS) were added into each well respectively. The mixture was incubated at 37° C.+5% CO₂ for 48 hours. Cells were centrifuged at 1500 rpm for 5 minutes. The supernatants were collected for ELISA assay to determine cytokine release. Cell pellet was resuspended in FACS buffer and stained with surface markers and the fixable L/D dye Zombie Aqua (Invitrogen). The staining panels included PEcy7 CD8, APC CD25, and APCcy7 CD69. After surface marker and Zombie aqua staining, the cells were washed twice with FACS buffer and intraocular stain was conducted with kit from TONBO(TNB-0607-kit). 200 μl of Fix/perm buffer were separately added into different wells and the plate was incubated at 40° C. for 1 hour. The cells were centrifuged and supernatant was discarded. The cells were washed one time with Perm buffer. 50 μl Intracellular staining cocktail (PE Granzyme B and PCPcy5.5 Ki67) were separately added into different wells and the plate was kept at room temperature for 30 minutes. The cells were rinsed one time with Perm buffer. After washing one more time with FACS buffer, the cells were resuspended in 120 μl FACS buffer and analyzed in Cytek Flow cytometer. Target cells were gated by dye-CD8+ granzyme B+ and dye-CD8+Ki67+ cells. The results in FIG. 7 and FIG. 8 show that 23D8/TRX4 significantly increased the levels of granzyme B and Ki67 in CD8 T cells compared with other candidates.

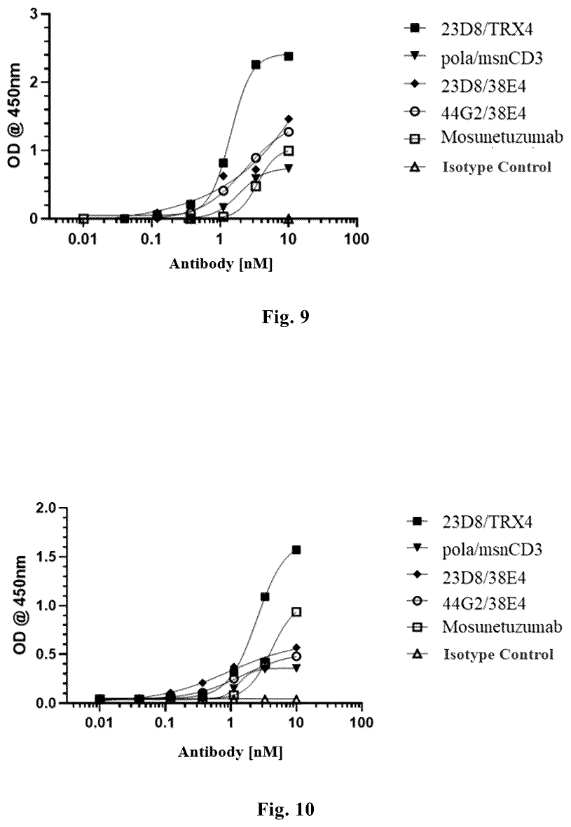

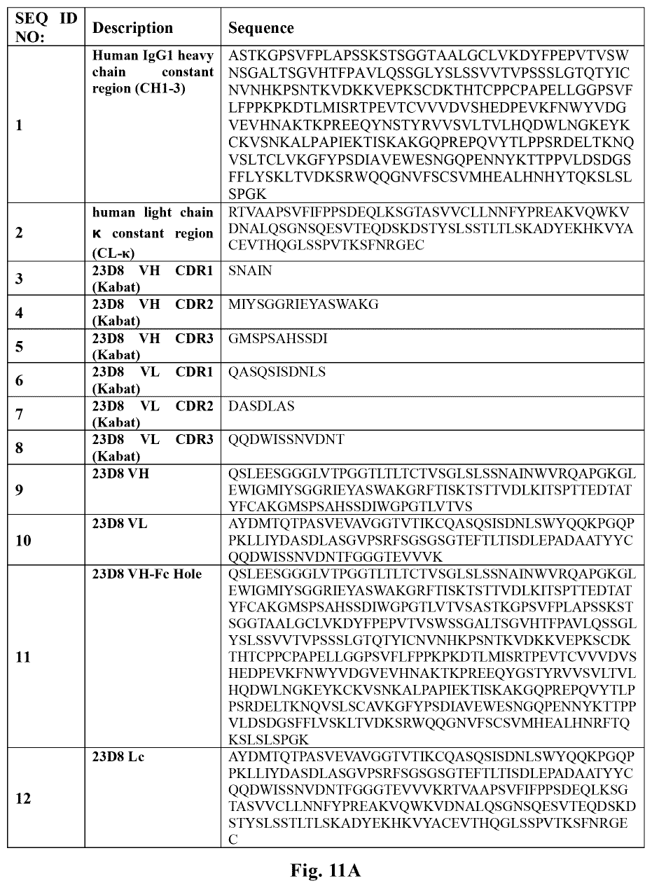

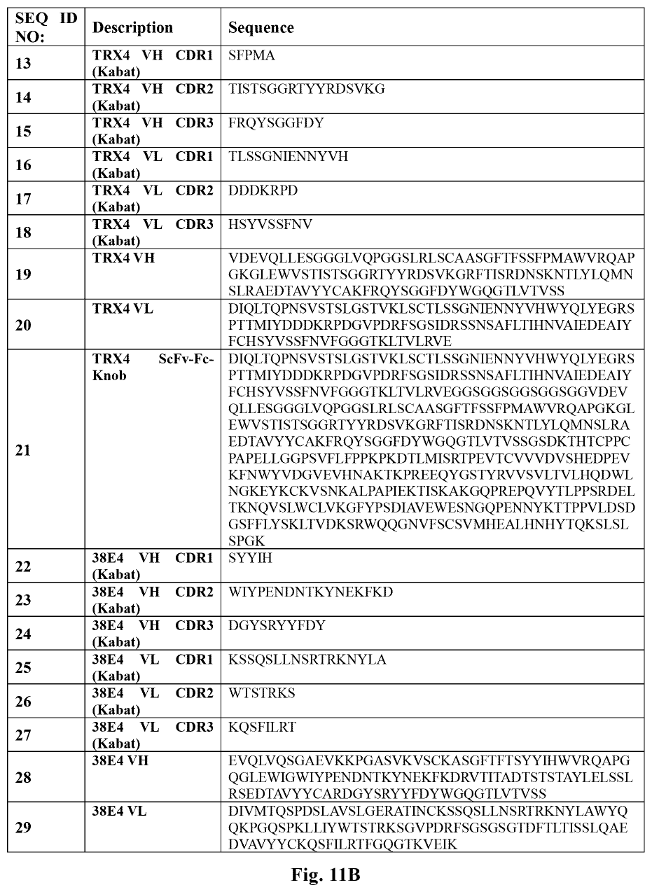

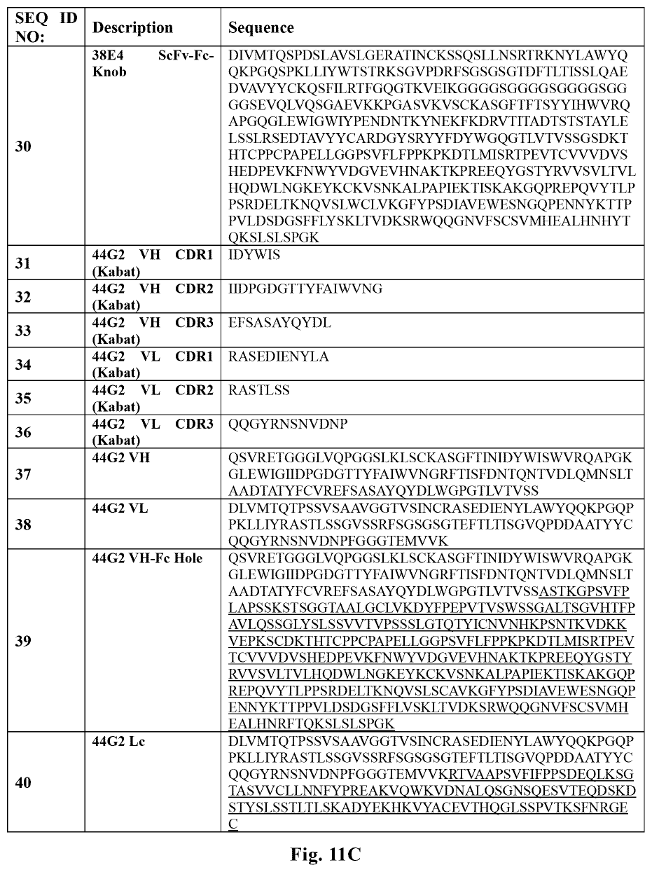

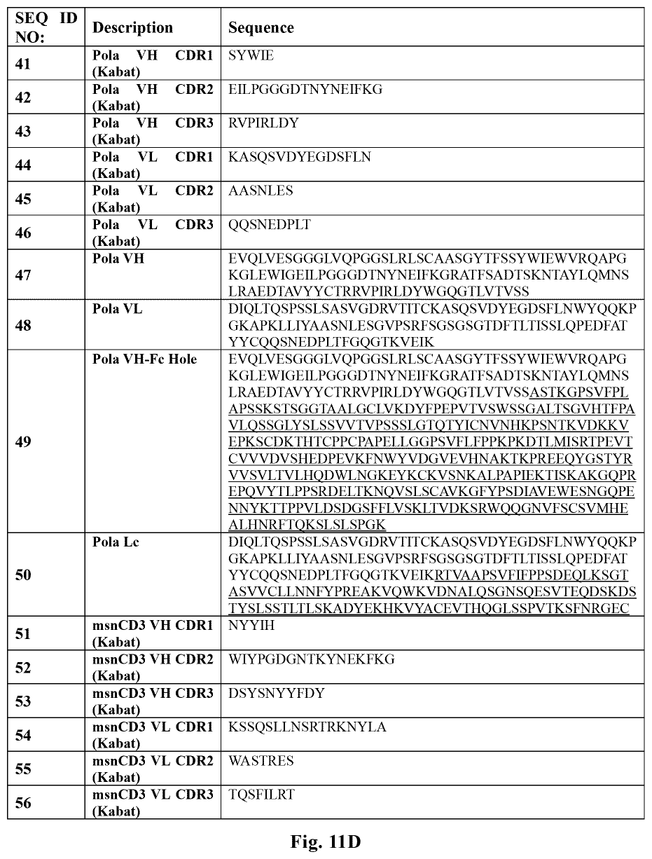

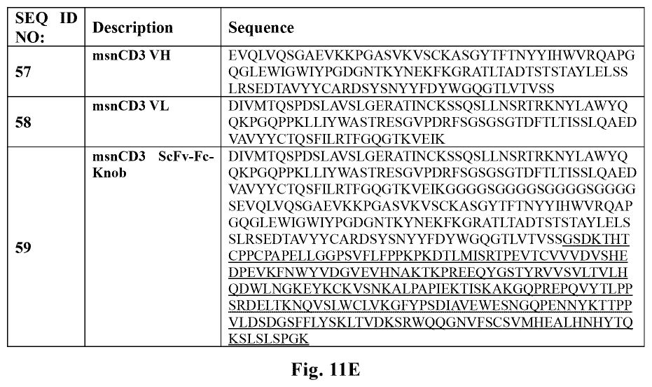

To examine the cytokine release induced by the CD79b/CD3 bispecific antibody, the supernatant from coculture system of CD8 T cell and BJAB tumor cell at 48 hours was collected for ELISA cytokine assay (IFNγ (invitrogen Kit Cat #88-7066) and TNFα(88-346-88)). High binding ELISA plates were coated with capture antibodies overnight. Next day, after washing 3 times with a wash buffer (PBST+0.05% Tween 20), 100 μl blocking buffer (1% BSA PBST) were separately added into different wells and plate was kept at room temperature for one hour. After washing 3 times with a wash buffer (PBST), 50 μl sample (5 times supernatant diluted)/well (50 μl 1% FBS PBS in blank wells) was added into the designated wells. The plate was sealed with plastic sealing film and incubated at room temperature for 2 hours. After incubation, the plate was washed 3 times with the wash buffer (PBST). Then, 100 μl of detection antibodies 23D8/TRX4, pola/msnCD3, 23D8/38E4, 44G2/38E4, Mosunetuzumab and negative control Human IgG Isotype Control (Invitrogen Catalog #02-7102) were separately added into different wells. The plate was sealed with a plastic sealing film and incubated for an additional hour at room temperature. After washing 3 times with wash buffer, 100 μl SP-avidin HRP was added to each well and the plate was incubated for an additional 30 min at room temperature. After washing 6 times with wash buffer, 100 μl TMB solution was added to each well and incubate the plate at room temperature for 15 minutes. Then, 100 μl stop solution was added to each well. Read the OD at 450 nm in the spectrometer. The results in FIG. 9 and FIG. 10 show that 23D8/TRX4 induces the highest TNFα and IFNγ production among the candidates tested.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A bispecific antibody, comprising: a first antigen-binding domain that specifically binds to a first antigen; and a second antigen-binding domain that specifically binds to a second antigen, wherein the first antigen is CD79b, wherein the second antigen is not CD79b;
wherein the first antigen-binding domain comprises a first heavy chain variable region (VH) and a first light chain variable region (VL), wherein,
the first VH comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and
the first VL comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 8.

2. The bispecific antibody of claim 1, wherein the first antigen binding domain comprises a first VH comprising the amino acid sequence set forth in SEQ ID NO: 9 or a variant thereof that comprises an amino acid sequence that is at least 80% identical to SEQ ID NO: 9, and a first VL comprising the amino acid sequence set forth in SEQ ID NO: 10 or a variant thereof that comprises an amino acid sequence that is at least 80% identical to SEQ ID NO: 10;
wherein, the variant comprises one or more amino acid substitutions, deletions or additions compared with original sequence; preferably, the substitution is a conservative substitution;
preferably, wherein the first antigen-binding domain comprises a first VH comprising the amino acid sequence set forth in SEQ ID NO: 9, and a first VL comprising the amino acid sequence set forth in SEQ ID NO: 10.

3. The bispecific antibody of claim 1, wherein the second antigen is selected from CD3, CD19, CD20, CD32B, CD137, CTLA-4 or BCMA. preferably, wherein the second antigen is CD3.

4. The bispecific antibody of claim 1, wherein the second antigen-binding domain comprises a second heavy chain variable region (VH) and a second light chain variable region (VL), wherein,
(1) the second VH comprises a CDR1 comprising the amino acid sequence set forth in SEQ ID NO: 13, a CDR2 comprising the amino acid sequence set forth in SEQ ID NO: 14, and a CDR3 comprising the amino acid sequence set forth in SEQ ID NO: 15; and the second VL comprises a CDR1 comprising the amino acid sequence set forth in SEQ ID NO: 16, a CDR2 comprising the amino acid sequence set forth in SEQ ID NO: 17, and a CDR3 comprising the amino acid sequence set forth in SEQ ID NO: 18;
(2) the second VH comprises a CDR1 comprising the amino acid sequence set forth in SEQ ID NO: 22, a CDR2 comprising the amino acid sequence set forth in SEQ ID NO: 23, and a CDR3 comprising the amino acid sequence set forth in SEQ ID NO: 24; and the second VL comprises a CDR1 comprising the amino acid sequence set forth in SEQ ID NO: 25, a CDR2 comprising the amino acid sequence set forth in SEQ ID NO: 26, and a CDR3 comprising the amino acid sequence set forth in SEQ ID NO: 27; or
(3) the second VH comprises a CDR1 comprising the amino acid sequence set forth in SEQ ID NO: 51, a CDR2 comprising the amino acid sequence set forth in SEQ ID NO: 52, and a CDR3 comprising the amino acid sequence set forth in SEQ ID NO: 53; and the second VL comprises a CDR1 comprising the amino acid sequence set forth in SEQ ID NO: 54, a CDR2 comprising the amino acid sequence set forth in SEQ ID NO: 55, and a CDR3 comprising the amino acid sequence set forth in SEQ ID NO: 56.

5. The bispecific antibody of claim 4, wherein,
(1) the second antigen-binding domain comprises a second VH comprising the amino acid sequence set forth in SEQ ID NO: 19 or a variant thereof that comprises an amino acid sequence that is at least 80% identical to SEQ ID NO: 19, and a second VL comprising the amino acid sequence set forth in SEQ ID NO: 20 or a variant thereof that comprises an amino acid sequence that is at least 80% identical to SEQ ID NO: 20;
(2) the second antigen-binding domain comprises a second VH comprising the amino acid sequence set forth in SEQ ID NO: 28 or a variant thereof that comprises an amino acid sequence that is at least 80% identical to SEQ ID NO: 28, and a second VL comprising the amino acid sequence set forth in SEQ ID NO: 29 or a variant thereof that comprises an amino acid sequence that is at least 80% identical to SEQ ID NO: 29; or
(3) the second antigen-binding domain comprises a second VH comprising the amino acid sequence set forth in SEQ ID NO: 57 or a variant thereof that comprises an amino acid sequence that is at least 80% identical to SEQ ID NO: 57, and a second VL comprising the amino acid sequence set forth in SEQ ID NO: 58 or a variant thereof that comprises an amino acid sequence that is at least 80% identical to SEQ ID NO: 58;
wherein, the variant comprises one or more amino acid substitutions, deletions or additions compared with original sequence; preferably, the substitution is a conservative substitution;
preferably, wherein the second antigen-binding domain comprises a second VH comprising the amino acid sequence set forth in SEQ ID NO: 19, and a second VL comprising the amino acid sequence set forth in SEQ ID NO: 20;
preferably, wherein the second antigen-binding domain comprises a second VH comprising the amino acid sequence set forth in SEQ ID NO: 28, and a second VL comprising the amino acid sequence set forth in SEQ ID NO: 29;
preferably, wherein the second antigen-binding domain comprises a second VH comprising the amino acid sequence set forth in SEQ ID NO: 57, and a second VL comprising the amino acid sequence set forth in SEQ ID NO: 58.

6. The bispecific antibody of claim 1, wherein the first antigen-binding domain and the second antigen-binding domain are independently selected from Fab, Fab′, (Fab′)₂, Fv, disulfide-linked Fv, scFv and single domain antibodies (sdAb);
alternatively, wherein the first antigen-binding domain or the second antigen-binding domain is rabbit derived, murine derived, fully humanized or chimeric;
preferably, wherein the first antigen-binding domain is Fab;
preferably, wherein the second antigen-binding domain is scFv, and the VH of the second antigen-binding domain is linked to the N-terminus or C-terminus of the VL of the second antigen-binding domain directly or by a peptide linker;
preferably, wherein the peptide linker is (GmS)n, and m and n are independently an integer not less than 0, such as 1, 2, 3 or 4 independently.

7. The bispecific antibody of claim 1, wherein the bispecific antibody further comprises an immunoglobulin Fc fragment;
preferably, wherein the immunoglobulin Fc fragment is linked to the N-terminus and/or C-terminus of the first antigen-binding domain and the second antigen-binding domain directly or by a peptide linker;
preferably, wherein the immunoglobulin Fc fragment is the Fc fragment of human IgG (such as IgG1, IgG2, IgG3 or IgG4);
preferably, wherein the peptide linker is (GmS)n, m and n are independently an integer not less than 0, such as independently 1, 2, 3 or 4;
preferably, wherein the immunoglobulin Fc fragment comprises knob and hole mutations.

8. The bispecific antibody of claim 1, wherein the bispecific antibody comprises knob and hole mutations, comprising:
(1) a peptide chain I-A that sequentially comprises the first VL and light chain constant region (CL) of the first antigen-binding domain from the N-terminus to the C-terminus; preferably, the CL is derived from a human immunoglobulin κ or λ chain;
(2) a peptide chain I-B that sequentially comprises the first VH and heavy chain constant region (CH) of the first antigen-binding domain from the N-terminus to the C-terminus; preferably, the CH is derived from human immunoglobulin IgG, such as IgG1, IgG2, and IgG3 or IgG4;
(3) a peptide chain I-C that sequentially comprises the second VL of the second antigen-binding domain, the peptide linker, the second VH of the second antigen-binding domain, the peptide linker and the Fc fragment from the N-terminus to the C-terminus; preferably, the peptide linker is (GmS)n, m and n are integers not less than 0, such as 1, 2, 3 or 4; preferably, the Fc fragment is the Fc fragment of human IgG (such as IgG1, IgG2, IgG3 or IgG4);
preferably, wherein the CH3 domain of the Fc fragment of the peptide chain I-B comprises a hole mutation, and the CH3 domain of the Fc fragment of the peptide chain I-C comprises a knob mutation;
preferably, wherein the peptide chain I-A comprises the amino acid sequence set forth in SEQ ID NO: 12; the peptide chain I-B comprises the amino acid sequence set forth in SEQ ID NO: 11; and the peptide chain I-C comprises the amino acid sequence set forth in any one of SEQ ID NOs: 21, 30 and 59.

9. An isolated nucleic acid molecule thereof encoding the bispecific antibody of claim 1.

10. A vector thereof comprising the nucleic acid molecule of claim 9; preferably, wherein the vector is a cloning vector or an expression vector.

11. A host cell thereof comprising the nucleic acid molecule of claim 9.

12. A method for preparing a bispecific antibody, comprising the following steps:
culturing the host cell of claim 11 under conditions that allow protein expression, and recovering the bispecific antibody from the host cell culture.

13. A conjugate comprising the bispecific antibody of claim 1 and a coupling moiety;
preferably, wherein the coupling moiety is selected from protein tags, such as purification tags; detectable labels, such as enzymes (such as horseradish peroxidase), radionuclides, fluorescent dyes, luminescent substances (such as chemiluminescent substances) or biotin; therapeutic agent, such as an anti-tumor drug; or another biologically active polypeptide.

14. A pharmaceutical composition thereof comprising the bispecific antibody of claim 1 and one or more pharmaceutically acceptable excipients;
preferably, wherein the pharmaceutical composition further comprises an additional anti-tumor drug.

15. A method of preventing and/or treating CD79b-related and/or CD3-related diseases in a subject, comprising administering to the subject an effective amount of the bispecific antibody of claim 1;
preferably, wherein the CD79b-related disease is a B-cell lymphoma-related disease, such as diffuse large B-cell lymphoma, acute B-cell leukemia, chronic lymphocytic leukemia, B-cell prelymphocytic leukemia, spleen with villous lymphocytes Lymphoma, hairy cell leukemia, follicular lymphoma, and mantle cell lymphoma;
preferably, wherein the CD3-related disease is an inflammatory disease or an autoimmune disease;
preferably, wherein the subject is a mammal, such as a human;
preferably, wherein the bispecific antibody, isolated nucleic acid molecule, vector, host cell, conjugate or pharmaceutical composition is used alone or in combination with another anti-tumor agent.

16. A method for detecting the presence or levels of CD79b and/or CD3 in a sample, comprising contacting the sample with the bispecific antibody of claim 1;
preferably, the method is an immunological detection, such as an immunoblot, an enzyme immunoassay (e.g., ELISA), a chemiluminescent immunoassay, a fluorescent immunoassay or a radioimmunoassay.

17. A method of diagnosing a subject of having a CD79b-related and/or CD3-related diseases comprising contacting a sample from the subject with the bispecific antibody of claim 1.

18. A multispecific antibody, comprising a first antigen-binding domain that specifically binds to a first antigen; and a second antigen-binding domain that specifically binds to a second antigen, wherein the first antigen is CD79b, wherein the second antigen is not CD79b,
wherein the first antigen-binding domain comprises a first heavy chain variable region (VH) and a first light chain variable region (VL), wherein
the first VH comprises a CDR1, a CDR2, and a CDR3 that are identical to CDR1, CDR2, and CDR3 that are present in SEQ ID NO: 9; and
the first VL comprises a CDR1, a CDR2, and a CDR3 that are identical to CDR1, CDR2, and CDR3 that are present in SEQ ID NO: 10,
preferably the second antigen is selected from CD3, CD19, CD20, CD32B, CD137, CTLA-4 or BCMA.

19. The multispecific antibody of claim 18, wherein the second antigen-binding domain comprises a heavy chain variable region (VH) and a light chain variable region (VL), wherein,
(1) the second VH comprises a CDR1, a CDR2, and a CDR3 that are identical to CDR1, CDR2, and CDR3 that are present in SEQ ID NO: 19; and
the second VL comprises a CDR1, a CDR2, and a CDR3 that are identical to CDR1, CDR2, and CDR3 that are present in SEQ ID NO: 20;
(2) the second VH comprises a CDR1, a CDR2, and a CDR3 that are identical to CDR1, CDR2, and CDR3 that are present in SEQ ID NO: 28; and
the second VL comprises a CDR1, a CDR2, and a CDR3 that are identical to CDR1, CDR2, and CDR3 that are present in SEQ ID NO: 29;
(3) the second VH comprises a CDR1, a CDR2, and a CDR3 that are identical to CDR1, CDR2, and CDR3 that are present in SEQ ID NO: 57; and
the second VL comprises a CDR1, a CDR2, and a CDR3 that are identical to CDR1, CDR2, and CDR3 that are present in SEQ ID NO: 58.

20. The multispecific antibody of claim 18, wherein the multispecific antibody is a bispecific antibody, wherein optionally
the first antigen-binding domain and the second antigen-binding domain are independently selected from Fab, Fab′, (Fab′)₂, Fv, disulfide-linked Fv, scFv and single domain antibodies (sdAb);
alternatively, wherein the first antigen-binding domain or the second antigen-binding domain is rabbit derived, murine derived, fully humanized or chimeric;
preferably, wherein the first antigen-binding domain is Fab;
preferably, wherein the second antigen-binding domain is scFv, and the VH of the second antigen-binding domain is linked to the N-terminus or C-terminus of the VL of the second antigen-binding domain directly or by a peptide linker;
preferably, wherein the peptide linker is (GmS)n, and m and n are independently an integer not less than 0, such as 1, 2, 3 or 4 independently.


SEQUENCE LISTING (59 sequences)

SEQ ID NO: 1 - protein, 330 aa, synthetic construct [sequence omitted]

SEQ ID NO: 2 - protein, 107 aa, synthetic construct [sequence omitted]

SEQ ID NO: 3 - protein, 5 aa, synthetic construct
  source 1..5: note = 23D8 VH CDR1 (Kabat)
SNAIN

SEQ ID NO: 4 - protein, 16 aa, synthetic construct
  source 1..16: note = 23D8 VH CDR2 (Kabat)
MIYSGGRIEYASWAKG

SEQ ID NO: 5 - protein, 11 aa, synthetic construct
  source 1..11: note = 23D8 VH CDR3 (Kabat)
GMSPSAHSSDI

SEQ ID NO: 6 - protein, 11 aa, synthetic construct
  source 1..11: note = 23D8 VL CDR1 (Kabat)
QASQSISDNLS

SEQ ID NO: 7 - protein, 7 aa, synthetic construct
  source 1..7: note = 23D8 VL CDR2 (Kabat)
DASDLAS

SEQ ID NO: 8 - protein, 12 aa, synthetic construct
  source 1..12: note = 23D8 VL CDR3 (Kabat)
QQDWISSNVDNT

SEQ ID NO: 9 - protein, 115 aa, synthetic construct
  source 1..115: note = 23D8 VH
QSLEESGGGLVTPGGTLTLTCTVSGLSLSSNAINWVRQAPGKGLEWIGMIYSGGRIEYASWAKGRFTISKTSTTVDLKITSPTTEDTATYFCAKGMSPSAHSSDIWGPGTLVTVS

SEQ ID NO: 10 - protein, 110 aa, synthetic construct
  source 1..110: note = 23D8 VL
AYDMTQTPASVEVAVGGTVTIKCQASQSISDNLSWYQQKPGQPPKLLIYDASDLASGVPSRFSGSGSGTEFTLTISDLEPADAATYYCQQDWISSNVDNTFGGGTEVVVK

SEQ ID NO: 11 - protein, 445 aa, synthetic construct
  source 1..445: note = 23D8 VH-Fc Hole
QSLEESGGGLVTPGGTLTLTCTVSGLSLSSNAINWVRQAPGKGLEWIGMIYSGGRIEYASWAKGRFTISKTSTTVDLKITSPTTEDTATYFCAKGMSPSAHSSDIWGPGTLVTVSASTKGPSVFPLAPSSKSTSGGTAALGCLVKDYFPEPVTVSWSSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 12 - protein, 217 aa, synthetic construct
  source 1..217: note = 23D8 Lc
AYDMTQTPASVEVAVGGTVTIKCQASQSISDNLSWYQQKPGQPPKLLIYDASDLASGVPSRFSGSGSGTEFTLTISDLEPADAATYYCQQDWISSNVDNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 5 aa, synthetic construct
  source 1..5: note = TRX4 VH CDR1 (Kabat)
SFPMA

SEQ ID NO: 14 - protein, 17 aa, synthetic construct
  source 1..17: note = TRX4 VH CDR2 (Kabat)
TISTSGGRTYYRDSVKG

SEQ ID NO: 15 - protein, 10 aa, synthetic construct
  source 1..10: note = TRX4 VH CDR3 (Kabat)
FRQYSGGFDY

SEQ ID NO: 16 - protein, 13 aa, synthetic construct
  source 1..13: note = TRX4 VL CDR1 (Kabat)
TLSSGNIENNYVH

SEQ ID NO: 17 - protein, 7 aa, synthetic construct
  source 1..7: note = TRX4 VL CDR2 (Kabat)
DDDKRPD

SEQ ID NO: 18 - protein, 9 aa, synthetic construct
  source 1..9: note = TRX4 VL CDR3 (Kabat)
HSYVSSFNV

SEQ ID NO: 19 - protein, 121 aa, synthetic construct
  source 1..121: note = TRX4 VH
VDEVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSS

SEQ ID NO: 20 - protein, 113 aa, synthetic construct
  source 1..113: note = TRX4 VL
DIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLRVE

SEQ ID NO: 21 - protein, 477 aa, synthetic construct
  source 1..477: note = TRX4 ScFv-Fc-Knob
DIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLRVEGGSGGSGGSGGSGGVDEVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 5 aa, synthetic construct
  source 1..5: note = 38E4 VH CDR1 (Kabat)
SYYIH

SEQ ID NO: 23 - protein, 17 aa, synthetic construct
  source 1..17: note = 38E4 VH CDR2 (Kabat)
WIYPENDNTKYNEKFKD

SEQ ID NO: 24 - protein, 10 aa, synthetic construct
  source 1..10: note = 38E4 VH CDR3 (Kabat)
DGYSRYYFDY

SEQ ID NO: 25 - protein, 17 aa, synthetic construct
  source 1..17: note = 38E4 VL CDR1 (Kabat)
KSSQSLLNSRTRKNYLA

SEQ ID NO: 26 - protein, 7 aa, synthetic construct
  source 1..7: note = 38E4 VL CDR2 (Kabat)
WTSTRKS

SEQ ID NO: 27 - protein, 8 aa, synthetic construct
  source 1..8: note = 38E4 VL CDR3 (Kabat)
KQSFILRT

SEQ ID NO: 28 - protein, 119 aa, synthetic construct
  source 1..119: note = 38E4 VH
EVQLVQSGAEVKKPGASVKVSCKASGFTFTSYYIHWVRQAPGQGLEWIGWIYPENDNTKYNEKFKDRVTITADTSTSTAYLELSSLRSEDTAVYYCARDGYSRYYFDYWGQGTLVTVSS

SEQ ID NO: 29 - protein, 112 aa, synthetic construct
  source 1..112: note = 38E4 VL
DIVMTQSPDSLAVSLGERATINCKSSQSLLNSRTRKNYLAWYQQKPGQSPKLLIYWTSTRKSGVPDRFSGSGSGTDFTLTISSLQAEDVAVYYCKQSFILRTFGQGTKVEIK

SEQ ID NO: 30 - protein, 480 aa, synthetic construct
  source 1..480: note = 38E4 ScFv-Fc-Knob
DIVMTQSPDSLAVSLGERATINCKSSQSLLNSRTRKNYLAWYQQKPGQSPKLLIYWTSTRKSGVPDRFSGSGSGTDFTLTISSLQAEDVAVYYCKQSFILRTFGQGTKVEIKGGGGSGGGGSGGGGSGGGGSEVQLVQSGAEVKKPGASVKVSCKASGFTFTSYYIHWVRQAPGQGLEWIGWIYPENDNTKYNEKFKDRVTITADTSTSTAYLELSSLRSEDTAVYYCARDGYSRYYFDYWGQGTLVTVSSGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 6 aa, synthetic construct
  source 1..6: note = 44G2 VH CDR1 (Kabat)
IDYWIS

SEQ ID NO: 32 - protein, 17 aa, synthetic construct
  source 1..17: note = 44G2 VH CDR2 (Kabat)
IIDPGDGTTYFAIWVNG

SEQ ID NO: 33 - protein, 11 aa, synthetic construct
  source 1..11: note = 44G2 VH CDR3 (Kabat)
EFSASAYQYDL

SEQ ID NO: 34 - protein, 11 aa, synthetic construct
  source 1..11: note = 44G2 VL CDR1 (Kabat)
RASEDIENYLA

SEQ ID NO: 35 - protein, 7 aa, synthetic construct
  source 1..7: note = 44G2 VL CDR2 (Kabat)
RASTLSS

SEQ ID NO: 36 - protein, 12 aa, synthetic construct
  source 1..12: note = 44G2 VL CDR3 (Kabat)
QQGYRNSNVDNP

SEQ ID NO: 37 - protein, 120 aa, synthetic construct
  source 1..120: note = 44G2 VH
QSVRETGGGLVQPGGSLKLSCKASGFTINIDYWISWVRQAPGKGLEWIGIIDPGDGTTYFAIWVNGRFTISFDNTQNTVDLQMNSLTAADTATYFCVREFSASAYQYDLWGPGTLVTVSS

SEQ ID NO: 38 - protein, 110 aa, synthetic construct
  source 1..110: note = 44G2 VL
DLVMTQTPSSVSAAVGGTVSINCRASEDIENYLAWYQQKPGQPPKLLIYRASTLSSGVSSRFSGSGSGTEFTLTISGVQPDDAATYYCQQGYRNSNVDNPFGGGTEMVVK

SEQ ID NO: 39 - protein, 450 aa, synthetic construct
  source 1..450: note = 44G2 VH-Fc Hole
QSVRETGGGLVQPGGSLKLSCKASGFTINIDYWISWVRQAPGKGLEWIGIIDPGDGTTYFAIWVNGRFTISFDNTQNTVDLQMNSLTAADTATYFCVREFSASAYQYDLWGPGTLVTVSSASTKGPSVFPLAPSSKSTSGGTAALGCLVKDYFPEPVTVSWSSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 40 - protein, 217 aa, synthetic construct
  source 1..217: note = 44G2 Lc
DLVMTQTPSSVSAAVGGTVSINCRASEDIENYLAWYQQKPGQPPKLLIYRASTLSSGVSSRFSGSGSGTEFTLTISGVQPDDAATYYCQQGYRNSNVDNPFGGGTEMVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 5 aa, synthetic construct
  source 1..5: note = Pola VH CDR1 (Kabat)
SYWIE

SEQ ID NO: 42 - protein, 17 aa, synthetic construct
  source 1..17: note = Pola VH CDR2 (Kabat)
EILPGGGDTNYNEIFKG

SEQ ID NO: 43 - protein, 8 aa, synthetic construct
  source 1..8: note = Pola VH CDR3 (Kabat)
RVPIRLDY

SEQ ID NO: 44 - protein, 15 aa, synthetic construct
  source 1..15: note = Pola VL CDR1 (Kabat)
KASQSVDYEGDSFLN

SEQ ID NO: 45 - protein, 7 aa, synthetic construct
  source 1..7: note = Pola VL CDR2 (Kabat)
AASNLES

SEQ ID NO: 46 - protein, 9 aa, synthetic construct
  source 1..9: note = Pola VL CDR3 (Kabat)
QQSNEDPLT

SEQ ID NO: 47 - protein, 117 aa, synthetic construct [sequence omitted]

SEQ ID NO: 48 - protein, 111 aa, synthetic construct [sequence omitted]

SEQ ID NO: 49 - protein, 447 aa, synthetic construct
  source 1..447: note = Pola VH-Fc Hole
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWSSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 50 - protein, 218 aa, synthetic construct
  source 1..218: note = Pola Lc
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 5 aa, synthetic construct
  source 1..5: note = msnCD3 VH CDR1 (Kabat)
NYYIH

SEQ ID NO: 52 - protein, 17 aa, synthetic construct
  source 1..17: note = msnCD3 VH CDR2 (Kabat)
WIYPGDGNTKYNEKFKG

SEQ ID NO: 53 - protein, 10 aa, synthetic construct
  source 1..10: note = msnCD3 VH CDR3 (Kabat)
DSYSNYYFDY

SEQ ID NO: 54 - protein, 17 aa, synthetic construct
  source 1..17: note = msnCD3 VL CDR1 (Kabat)
KSSQSLLNSRTRKNYLA

SEQ ID NO: 55 - protein, 7 aa, synthetic construct
  source 1..7: note = msnCD3 VL CDR2 (Kabat)
WASTRES

SEQ ID NO: 56 - protein, 8 aa, synthetic construct
  source 1..8: note = msnCD3 VL CDR3 (Kabat)
TQSFILRT

SEQ ID NO: 57 - protein, 119 aa, synthetic construct
  source 1..119: note = msnCD3 VH
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSS

SEQ ID NO: 58 - protein, 112 aa, synthetic construct
  source 1..112: note = msnCD3 VL
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 59 - protein, 480 aa, synthetic construct
  source 1..480: note = msnCD3 ScFv-Fc-Knob
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKGGGGSGGGGSGGGGSGGGGSEVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSSGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK